(19) United States
(12) Patent Application Publication
(10) Pub. No.: US 2007/0071797 A1
(43) Pub. Date: Mar. 29, 2007

(54) Lotioned fibrous structures

(76) Inventors: Diego Antonio Hernandez-Munoa, Tuxtla Gutierrez (MX); Scott Thomas Loughran, West Chester, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/522,267

(22) Filed: Sep. 14, 2006

Related U.S. Application Data
(60) Provisional application No. 60/718,068, filed on Sep. 16, 2005.

(51) Int. Cl. A61K 9/70

(52) U.S. Cl. 424/443

(57) ABSTRACT

Lotioned fibrous structures and sanitary tissue products comprising such fibrous structures are provided. More particularly, fibrous structures comprising two or more different compositions arranged on a surface of the fibrous structure such that one of the compositions is sandwiched between the fibrous structure surface and the other composition is provided. Sanitary tissue products comprising such fibrous structures and methods for making such fibrous structures are also provided.

### CROSS REFERENCE TO RELATED APPLICATION

This application claims the benefit of U.S. Provisional Application No. 60/718,068, filed Sep. 16, 2005.

### FIELD OF THE INVENTION

The present invention relates to fibrous structures and sanitary tissue products comprising such fibrous structures. More particularly, the present invention relates to fibrous structures comprising two or more different compositions arranged on a surface of the fibrous structure such that one of the compositions is sandwiched between the fibrous structure surface and the other composition and/or the compositions are phase registered with one another on the fibrous structure surface, sanitary tissue products comprising same and methods for making same.

### BACKGROUND OF THE INVENTION

Lotioned fibrous structures are known in the art. For example, formulators have developed costly and time-consuming ways to blend lotion compositions and surface treating compositions into a single composition and then subsequently apply such a single composition to the surface of a fibrous structure. The resulting fibrous structure comprises a surface that comprises a single composition, not two different compositions. By doing so, certain performance properties of the fibrous structure are enhanced at the expense of other performance properties of the fibrous structure.

Other formulators of lotioned fibrous structures have applied a single lotion composition to a surface of a fibrous structure.

Still other formulators have applied a single surface treating composition to a fibrous structure without subsequently adding a lotion composition.

Lotion compositions and surface treating compositions of the prior art are inherently incompatible.

Accordingly, there is a long felt need to identify and develop fibrous structures that contain two or more different compositions, especially two or more compositions, such as a surface treating composition and a lotion composition, wherein application of the compositions is intelligently designed to provide maximum benefit with minimum waste and/or cost.

### SUMMARY OF THE INVENTION

It has been found that equal performance as compared to prior art fibrous structures can be obtained if two or more different compositions are phase registrably applied to a surface of a fibrous structure such that the two or more different compositions cover less than the entire surface area of a surface of the fibrous structure. Further, by doing so, skin benefit agents may be applied in a relatively thin layer of a composition that is capable of being transferred to a user's skin during use. In other words, the skin benefit agent is selectively placed on a fibrous structure to optimize its performance without unnecessary waste.

In one example of the present invention, a fibrous structure comprising a surface comprising two or more different compositions, especially separate and/or discrete compositions, is provided.

In another example of the present invention, a fibrous structure comprising a surface comprising two or more different compositions wherein at least two of the two or more different compositions are phase registered.

In even another example of the present invention, a fibrous structure comprising a surface comprising a first region comprising a surface treating composition and a lotion composition and a second region being void of at least one of the surface treating composition and the lotion composition, especially wherein the second region is surrounded by at least two of the first regions, is provided.

In still another example of the present invention, a fibrous structure comprising a surface comprising a first composition and a second composition that is incompatible with the first composition, wherein the first and second compositions are phase registered, is provided.

In yet another example of the present invention, a process for treating a surface of a fibrous structure, the process comprising the steps of:
a. applying a first composition to the surface of the fibrous structure;
b. applying a second composition to the first composition; and
c. applying a third composition to the second composition, is provided.

In even another example of the present invention, a process for treating a surface of a fibrous structure, the process comprising the steps of:
a. applying a first composition to the surface of the fibrous structure; and
b. phase registrably applying a second composition to the first composition, is provided.

In even yet another example of the present invention, a single- or multi-ply sanitary tissue product comprising a fibrous structure according to the present invention is provided.

Accordingly, the present invention provides fibrous structures comprising two or more different compositions, processes for treating fibrous structures to produce such fibrous structures comprising two or more different compositions, and sanitary tissue products comprising such fibrous structures.

### BRIEF DESCRIPTION OF THE DRAWINGS

FIG. 1 is a schematic representation of a fibrous structure according to the present invention;

FIG. 2 is a cross-sectional view taken along line 2-2 of FIG. 1;

FIG. 3 is a side view of an untreated fibrous structure according to the present invention.

### DETAILED DESCRIPTION OF THE INVENTION

Definitions

“Different compositions” as used herein means that one composition comprises an ingredient that is not present in another composition and/or one composition is physically different than another composition (for example, one composition is a liquid at a given temperature and another composition is a solid at that given temperature).

“Compatible” as used herein means that two or more different compositions are able to exist in close and permanent association for an indefinite period (for example greater than 2 hours and/or greater than 12 hours and/or greater than 24 hours and/or greater than 7 days and/or greater than 4 weeks). For example, two or more different liquid compositions are compatible if they are miscible in all proportions and do not undergo phase separation on standing. A liquid composition and a solid composition are compatible if the solid is soluble in the liquid, but not otherwise. Two or more different solid compositions are compatible if they can exist in intimate contact for long periods (for example greater than 2 hours and/or greater than 12 hours and/or greater than 24 hours and/or greater than 7 days and/or greater than 4 weeks) of time with no adverse effect of one on the other.

“Incompatible” as used herein means that two or more different compositions are not able to exist in close and permanent association for an indefinite period of time (for example, the opposite of “compatible” as described above).

“Miscible” as used herein means that a liquid composition is able to dissolve uniformly (without phase separation) in a different liquid composition and remain dissolved uniformly for an indefinite period of time (for example greater than 2 hours and/or greater than 12 hours and/or greater than 24 hours and/or greater than 7 days and/or greater than 4 weeks). For example, a liquid composition is miscible in a different liquid composition if it is miscible in the different liquid composition at least 5% and/or at least 10% and/or at least 30% and/or at least 50% and/or at least 75% and/or at least 90% and/or at 100% by weight at 25° C.

“Soluble” as used herein means that one composition blends uniformly with a different composition. “Miscible” is synonymous with “soluble” with respect to different liquid compositions. However, with respect to different compositions where one is a solid composition and the other is a liquid composition, the term “soluble” is used to describe the properties of the solid composition within the liquid composition. For example, a solid composition is soluble in a liquid composition if it is soluble in the liquid composition at least 5% and/or at least 10% and/or at least 30% and/or at least 50% and/or at least 75% and/or at least 90% and/or at 100% by weight at 25° C. In another example, a solid composition is soluble in a liquid composition if the solid composition doesn't phase separate from the liquid composition at 25° C. after greater than 2 hours and/or greater than 12 hours and/or greater than 24 hours and/or greater than 7 days and/or greater than 4 weeks.

“Phase registered” as used herein means that two or more different compositions are arranged such that the two or more different compositions cover the identical amount of surface area, wherein one composition is on top of the other composition. In one example, two different compositions are phase registered if the first composition covers 100% of the surface of a fibrous structure, and the second composition covers 100% of the surface of the composition of the fibrous structure (the first composition being positioned between the second composition and the surface of the fibrous structure). In another example, two different compositions are phase registered if the first composition covers less than 100% of the surface of a fibrous structure (such as 50% of the surface of the fibrous structure), and the second composition covers the same amount of surface area as the first composition (the first composition being positioned between the second composition and the surface of the fibrous structure).

“Fiber” as used herein means an elongate particulate having an apparent length greatly exceeding its apparent diameter, i.e. a length to diameter ratio of at least about 10. Fibers having a non-circular cross-section are common; the “diameter” in this case may be considered to be the diameter of a circle having cross-sectional area equal to the cross-sectional area of the fiber. More specifically, as used herein, “fiber” refers to papermaking fibers. The present invention contemplates the use of a variety of papermaking fibers, such as, for example, natural fibers or synthetic fibers, or any other suitable fibers, and any combination thereof.

Natural papermaking fibers useful in the present invention include animal fibers, mineral fibers, plant fibers and mixtures thereof. Animal fibers may, for example, be selected from the group consisting of: wool, silk and mixtures thereof. Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered fibrous structure. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. Softwood fibers derived from the kraft process and originating from more-northern climates may be preferred. These are often referred to as northern softwood kraft (NSK) pulps.

Synthetic fibers may be selected from the group consisting of: wet spun fibers, dry spun fibers, melt spun (including melt blown) fibers, synthetic pulp fibers and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

“Fibrous structure” as used herein means a structure that comprises one or more fibers. Nonlimiting examples of processes for making fibrous structures include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure is formed, drying and/or bonding the fibers together such that a fibrous structure is formed, and/or further processing the fibrous structure such that a finished fibrous structure is formed. For example, in typical papermaking processes, the finished fibrous structure is the fibrous structure that is wound on the reel at the end of papermaking, but before converting thereof into a sanitary tissue product.

Nonlimiting types of fibrous structures according to the present invention include conventionally felt-pressed fibrous structures; pattern densified fibrous structures; and high-bulk, uncompacted fibrous structures. The fibrous structures may be of a homogeneous or multilayered (two or three or more layers) construction; and the sanitary tissue products made therefrom may be of a single-ply or multi-ply construction.

The fibrous structures may be post-processed, such as by embossing and/or calendaring and/or folding and/or printing images thereon.

The fibrous structures may be through-air-dried fibrous structures or conventionally dried fibrous structures.

The fibrous structures may be creped or uncreped.

“Sanitary tissue product” comprises one or more fibrous structures, converted or not, that is useful as a wiping implement for post-urinary and post-bowel movement cleaning (toilet tissue), for otorhinolaryngological discharges (facial tissue and/or disposable handkerchiefs), and multi-functional absorbent and cleaning uses (absorbent towels and/or wipes). In one example, a lotion composition-containing multi-ply disposable handkerchief having a caliper of from about 0.1 mm to about 0.4 mm in accordance with the present invention is provided.

“Ply” or “Plies” as used herein means an individual finished fibrous structure optionally to be disposed in a substantially contiguous, face-to-face relationship with other plies, forming a multiple ply finished fibrous structure product and/or sanitary tissue product. It is also contemplated that a single fibrous structure can effectively form two “plies” or multiple “plies”, for example, by being folded on itself.

“Surface of a fibrous structure” as used herein means that portion of the fibrous structure that is exposed to the external environment. In other words, the surface of a fibrous structure is that portion of the fibrous structure that is not completely surrounded by other portions of the fibrous structure.

“User Contacting Surface” as used herein means that portion of the fibrous structure and/or surface treating composition and/or lotion composition present directly and/or indirectly on the surface of the fibrous structure that is exposed to the external environment. In other words, it is that surface formed by the fibrous structure including any surface treating composition and/or lotion composition present directly and/or indirectly on the surface of the fibrous structure that contacts an opposing surface when used by a user. For example, it is that surface formed by the fibrous structure including any surface treating composition and/or lotion composition present directly and/or indirectly on the surface of the fibrous structure that contacts a user's skin when a user wipes his/her skin with the fibrous structure of the present invention.

In one example, the user contacting surface, especially for a textured and/or structured fibrous structure, such as a through-air-dried fibrous structure and/or an embossed fibrous structure, may comprise raised areas and recessed areas of the fibrous structure. In the case of a through-air-dried, pattern densified fibrous structure the raised areas may be knuckles and the recessed areas may be pillows and vice versa. Accordingly, the knuckles may, directly and/or indirectly, comprise the surface treating composition and lotion composition and the pillows may be void of the surface treating composition and the lotion composition and vice versa so that when a user contacts the user's skin with the fibrous structure, only the lotion composition contacts the user's skin. A similar case is true for embossed fibrous structures where the embossed areas may, directly and/or indirectly, comprise the surface treating composition and the lotion composition and the non-embossed areas may be void of the surface treating composition and the lotion composition and vice versa.

The user contacting surface may be present on the fibrous structure and/or sanitary tissue product before use by the user and/or the user contacting surface may be created/formed prior to and/or during use of the fibrous structure and/or sanitary tissue product by the user, such as upon the user applying pressure to the fibrous structure and/or sanitary tissue product as the user contacts the user's skin with the fibrous structure and/or sanitary tissue product.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

Lotioned Fibrous Structure

The fibrous structure according to the present invention comprises a surface comprising two or more different compositions. In one example, the surface comprises at least three different compositions. In another example, at least two of the two or more different compositions are phase registered.

In one example, the surface of the fibrous structure may comprise a layer of a surface treating composition and a layer of a lotion composition.

In another example, the surface of the fibrous structure may comprise a layer of a surface treating composition and two layers of different lotion compositions.

The surface treating composition may not be visibly discernible as a layer on the surface of the fibrous structure since it has a tendency to migrate into the fibrous structure and/or coat the fibers of the fibrous structure, especially the outer fibers of the fibrous structure that make up the surface of the fibrous structure.

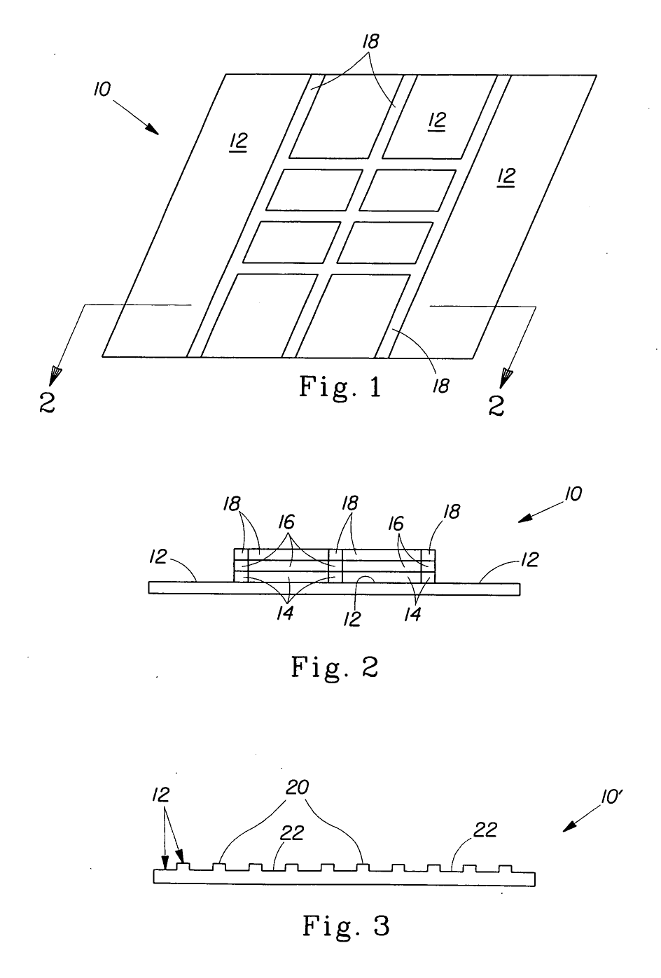

As shown in FIGS. 1 and 2, a fibrous structure 10 comprises a surface 12. The surface 12 comprises a surface treating composition 14, a first lotion composition 16 and optionally, a second lotion composition 18. The second lotion composition 18 is only visible in FIG. 1 due to the fact that the second lotion composition 18 and the first lotion composition 16 and the surface treating composition 14 are all phase registered on the surface 12 of the fibrous structure 10. The three different compositions may be present on the surface 12 of the fibrous structure 10 in the form of a pattern such that they cover less than the entire surface area of the surface 12 of the fibrous structure 10, as shown in FIGS. 1 and 2, or they may be present on the surface 12 such that they cover the entire or substantially the entire surface area of the surface 12 of the fibrous structure 10.

As shown in FIGS. 1 and 2, the surface 12 of the fibrous structure 10 comprises two regions, a first region that comprises a surface treating composition and/or a lotion composition and a second region being void of the surface treating composition and/or the lotion composition. In one example, the void regions comprise neither a surface treating composition nor a lotion composition.

As shown in FIG. 2, the fibrous structure 10 comprises two or more different regions that exhibit different elevations.

As shown in FIG. 1, the surface treating composition 14 and the first lotion composition 16 and optionally, the second lotion composition 18, both of which may be incompatible with the surface treating composition 14, are present on the surface 12 of the fibrous structure 10 in the form of a continuous network and the at least portions of the regions that are void of the surface treating composition 14 and/or the lotion compositions 16, 18 are present on the surface 12 of the fibrous structure 10 as discrete regions.

Alternatively, the void regions of the surface 12 of the fibrous structure 10 may be present on the fibrous structure 10 as a continuous network and the regions that comprise the surface treating composition 14 and/or the lotion compositions 16, 18 may be present on the surface 12 of the fibrous structure 10 as discrete regions.

In another example, the surface of the fibrous structure may comprise a first region that comprises three or more different compositions, especially phase registered different compositions, and a second region that is void of at least one of the three or more different compositions.

The second lotion composition 18 may comprise a skin benefit agent, which can be transferred to a user's skin during use. In one example, the second lotion composition 18 is less than 50% and/or less than 30% and/or less than 20% and/or less than 10% by weight of the first lotion composition 16.

The surface treating composition 14 and the lotion composition 16 may be applied to the surface 12 of the fibrous structure 10 by any suitable means known in the art.

Any contact or contact-free application suitable for applying the surface treating composition, such as spraying, dipping, padding, printing, slot extruding, such as in rows or patterns, rotogravure printing, flexographic printing, offset printing, screen printing, mask or stencil application process and mixtures thereof can be used to apply the surface treating composition and/or lotion composition to the surface of the fibrous structure and/or sanitary tissue product. Surface treating compositions can be applied to the fibrous structure and/or sanitary tissue product before, concurrently, or after the lotion composition application to the fibrous structure and/or sanitary tissue product.

In one example, the surface treating composition and/or the lotion composition is applied to the surface of the fibrous structure during the fibrous structure making process, such as before and/or after drying the fibrous structure.

In another example, the surface treating composition and/or the lotion composition is applied to the surface of the fibrous structure during the converting process.

In yet another example, the surface treating composition is applied to the surface of a fibrous structure prior to application of the lotion composition. The surface treating composition can be applied during papermaking and/or converting, especially if applied to the outside layer of a layered fibrous structure and/or sanitary tissue product comprising such layered fibrous structure.

The surface treating composition and lotion composition can be applied by separate devices or by a single device that has two or more chambers capable of separately delivering the different compositions, especially incompatible, different compositions, such as the surface treating composition and the lotion composition.

The application devices may be sequentially arranged along the papermaking (fibrous structure making) and/or converting process.

For example, if the fibrous structure 10′, as shown in FIG. 3, prior to being treated by the surface treating composition and/or the lotion composition, comprises regions of different elevation 20, 22, such as by being a patterned-densified and/or differentially densified fibrous structure (knuckles and pillows) or by being embossed or creped fibrous structure, then the surface treating composition 14 and/or the lotion composition 16 may be slot extruded and/or printed, such as gravure roll printing, onto one elevation 20 of the fibrous structure 10′ and not onto the other elevation 22 of the fibrous structure 10′.

Surface Treating Composition

A surface treating composition, for purposes of the present invention, is a composition that improves the tactile sensation of a surface of a fibrous structure perceived by a user whom holds a fibrous structure and/or sanitary tissue product comprising the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel.

The surface treating composition may or may not be transferable. Typically, it is substantially non-transferable.

The surface treating composition may increase or decrease the surface friction of the surface of the fibrous structure, especially the user contacting surface of the fibrous structure. Typically, the surface treating composition will reduce the surface friction of the surface of the fibrous structure compared to a surface of the fibrous structure without such surface treating composition.

The surface treating composition may have a wettability tension less than or equal to the surface tension of a lotion composition applied to a surface of a fibrous structure treated with the surface treating composition so as to minimize the spreading of the lotion composition that comes into contact with the surface treating composition and/or to reduce and/or inhibit migration of the lotion composition into the fibrous structure.

The surface treating composition comprises a surface treating agent. The surface treating composition during application to the fibrous structure may comprise at least about 0.1% and/or at least 0.5% and/or at least about 1% and/or at least about 3% and/or at least about 5% to about 90% and/or to about 80% and/or to about 70% and/or to about 50% and/or to about 40% by weight of the surface treating agent. In one example, the surface treating composition comprises from about 5% to about 40% by weight of the surface treating agent.

The surface treating composition present on the fibrous structure and/or sanitary tissue product comprising the fibrous structure of the present invention may comprise at least about 0.01% and/or at least about 0.05% and/or at least about 0.1% of total basis weight of the surface treating agent. In one example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the surface treating composition.

Nonlimiting examples of suitable surface treating agents can be selected from the group consisting of: polymers such as polyethylene and derivatives thereof, hydrocarbons, waxes, oils, silicones, organosilicones (oil compatible), quaternary ammonium compounds, fluorocarbons, substituted C₁₀-C₂₂ alkanes, substituted C₁₀-C₂₂ alkenes, in particular derivatives of fatty alcohols and fatty acids(such as fatty acid amides, fatty acid condensates and fatty alcohol condensates), polyols, derivatives of polyols (such as esters and ethers), sugar derivatives (such as ethers and esters), polyglycols (such as polyethyleneglycol) and mixtures thereof.

In one example, the surface treating composition of the present invention is a microemulsion and/or a macroemulsion of a surface treating agent (for example an aminofunctional polydimethylsiloxane, specifically an aminoethylaminopropyl polydimethylsiloxane) in water. In such an example, the concentration of the surface treating agent within the surface treating composition may be from about 3% to about 60% and/or from about 4% to about 50% and/or from about 5% to about 40%. A nonlimiting examples of such microemulsions are commercially available from Wacker Chemie (MR1003, MR103, MR102). A nonlimiting example of such a macroemulsion is commercially available from General Electric Silicones (CM849).

Nonlimiting examples of suitable waxes may be selected from the group consisting of: paraffin, polyethylene waxes, beeswax and mixtures thereof.

Nonlimiting examples of suitable oils may be selected from the group consisting of: mineral oil, silicone oil, silicone gels, petrolatum and mixtures thereof.

Nonlimiting examples of suitable silicones may be selected from the group consisting of: polydimethylsiloxanes, aminosilicones, cationic silicones, quaternary silicones, silicone betaines and mixtures thereof.

Nonlimiting examples of suitable polysiloxanes and/or monomeric/oligomeric units may be selected from the compounds having monomeric siloxane units of the following structure: 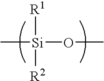
wherein, R¹ and R2, for each independent siloxane monomeric unit can each independently be hydrogen or any alkyl, aryl, alkenyl, alkaryl, arakyl, cycloalkyl, halogenated hydrocarbon, or other radical. Any of such radical can be substituted or unsubstituted. R¹ and R² radicals of any particular monomeric unit may differ from the corresponding functionalities of the next adjoining monomeric unit. Additionally, the polysiloxane can be either a straight chain, a branched chain or have a cyclic structure. The radicals R¹ and R² can additionally independently be other silaceous functionalities such as, but not limited to siloxanes, polysiloxanes, silanes, and polysilanes. The radicals R¹ and R² may contain any of a variety of organic functionalities including, for example, alcohol, carboxylic acid, phenyl, and amine functionalities. The end groups can be reactive (alkoxy or hydroxyl) or nonreactive (trimethylsiloxy). The polymer can be branched or unbranched.

In one example, suitable polysiloxanes include straight chain organopolysiloxane materials of the following general formula: 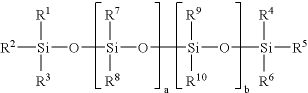
wherein each R¹-R⁹ radical can independently be any C₁-C₁₀ unsubstituted alkyl or aryl radical, and R¹⁰ of any substituted C₁-C₁₀ alkyl or aryl radical. In one example, each R¹-R⁹ radical is independently any C₁-C₄ unsubstituted alkyl group. Those skilled in the art will recognize that technically there is no difference whether, for example, R⁹ or R¹⁰ is the substituted radical. In another example, the mole ratio of b to (a+b) is between 0 and about 20% and/or between 0 and about 10% and/or between about 1% and about 5%.

A nonlimiting example of a cationic silicone polymer that can be used as a surface treating agent comprises one or more polysiloxane units, preferably polydimethylsiloxane units of formula —{(CH₃)₂SiO}_{c}— having a degree of polymerization, c, of from about 1 to about 1000 and/or from about 20 to about 500 and/or from about 50 to about 300 and/or from about 100 to about 200, and organosilicone-free units comprising at least one diquaternary unit. In one example, the cationic silicone polymer has from about 0.05 to about 1.0 and/or from about 0.2 to about 0.95 and/or from about 0.5 to about 0.9 mole fraction of the organosilicone-free units selected from cationic divalent organic moieties. The cationic divalent organic moiety may be selected from N,N,N′,N′-tetramethyl-1,6-hexanediammonium units.

The cationic silicone polymer may contain from about 0 to about 0.95 and/or from about 0.001 to about 0.5 and/or from about 0.05 to about 0.2 mole fraction of the total of organosilicone-free units, polyalkyleneoxide amines of the following formula:
[—Y—O(—CₐH₂ₐO)_{b}—Y—]
wherein Y is a divalent organic group comprising a secondary or tertiary amine, such as a C₁ to C₈ alkylenamine residue; a is from 2 to 4, and b is from 0 to 100.

Such polyalkyleneoxide amine-containing units can be obtained by introducing in the silicone polymer structure, compounds such as those sold under the tradename Jeffamine® from Huntsman Corporation. A preferred Jeffamine is Jeffamine ED-2003.

The cationic silicone polymer may contain from about 0 and/or from about 0.001 to about 0.2 mole fraction, of the total of organosilicone-free units, of —NR₃+ wherein R is alkyl, hydroxyalkyl or phenyl. These units can be thought of as end-caps.

Moreover the cationic silicone polymer generally contains anions, selected from inorganic and organic anions.

A nonlimiting example of a cationic silicone polymer comprises one or more polysiloxane units and one or more quaternary nitrogen moieties, and includes polymers wherein the cationic silicone polymer has the formula: 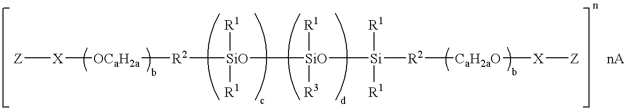
wherein:
R¹ is independently selected from the group consisting of: C₁₋₂₂ alkyl, C₂₋₂₂ alkenyl, C₆₋₂₂ alkylaryl, aryl, cycloalkyl, and mixtures thereof;
R² is independently selected from the group consisting of: divalent organic moieties that may contain one or more oxygen atoms (such moieties preferably consist essentially of C and H or of C, H and O);
X is independently selected from the group consisting of ring-opened epoxides;
R³ is independently selected from polyether groups having the formula:
   —M¹(CₐH₂ₐO)_{b}—M²
   wherein M¹ is a divalent hydrocarbon residue; M² is independently selected from the group consisting of H, C₁₋₂₂ alkyl, C₂₋₂₂ alkenyl, C₆₋₂₂ alkylaryl, aryl, cycloalkyl, C₁₋₂₂ hydroxyalkyl, polyalkyleneoxide, (poly)alkoxy alkyl, and mixtures thereof;
Z is independently selected from the group consisting of monovalent organic moieties comprising at least one quaternized nitrogen atom;
a is from 2 to 4; b is from 0 to 100; c is from 1 to 1000 and/or greater than 20 and/or greater than 50 and/or less than 500 and/or less than 300 and/or from 100 to 200;
d is from 0 to 100; n is the number of positive charges associated with the cationic silicone polymer, which is greater than or equal to 2; and A is a monovalent anion.

Another nonlimiting example of a cationic silicone polymer comprises one or more polysiloxane units and one or more quaternary nitrogen moieties, and includes polymers wherein the cationic silicone polymer has the formula: 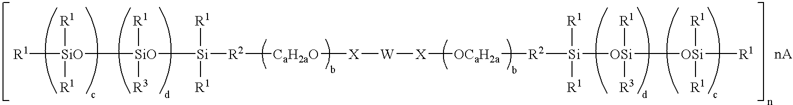
wherein:
R¹ is independently selected from the group consisting of: C₁₋₂₂ alkyl, C₂₋₂₂ alkenyl, C₆₋₂₂ alkylaryl, aryl, cycloalkyl, and mixtures thereof;
R² is independently selected from the group consisting of: divalent organic moieties that may contain one or more oxygen atoms;
X is independently selected from the group consisting of ring-opened epoxides;
R³ is independently selected from polyether groups having the formula:
   —M¹(CₐH₂ₐO)_{b}—M²
   wherein M¹ is a divalent hydrocarbon residue; M² is independently selected from the group-consisting of H, C₁₋₂₂ alkyl, C₂₋₂₂ alkenyl, C₆₋₂₂ alkylaryl, aryl, cycloalkyl, C₁₋₂₂ hydroxyalkyl, polyalkyleneoxide, (poly)alkoxy alkyl, and mixtures thereof;
X is independently selected from the group consisting of ring-opened epoxides;
W is independently selected from the group consisting of divalent organic moieties comprising at least one quaternized nitrogen atom;
a is from 2 to 4; b is from 0 to 100; c is from 1 to 1000 and/or greater than 20 and/or greater than 50 and/or less than 500 and/or less than 300 and/or from 100 to 200; d is from 0 to 100; n is the number of positive charges associated with the cationic silicone polymer, which is greater than or equal to 1; and A is a monovalent anion, in other words, a suitable counterion.

References disclosing nonlimiting examples of suitable polysiloxanes include U.S. Pat. Nos. 2,826,551, 3,964,500, 4,364,837, 5,059,282, 5,529,665, 5,552,020 and British Patent No. 849,433 and Silicone Compounds, pp. 181-217, distributed by Petrach Systems, Inc., which contains an extensive listing and description of polysiloxanes in general.

Viscosity of polysiloxanes useful for this invention may vary as widely as the viscosity of polysiloxanes in general vary, so long as the polysiloxane can be rendered into a form which can be applied to the fibrous structures herein. This includes, but is not limited to, viscosity as low as about 25 centistokes to about 20,000,000 centistokes or even higher.

Nonlimiting examples of suitable quaternary ammonium compounds may be selected from compounds having the formula: 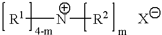
wherein:
m is 1 to 3; each R¹ is independently a C₁-C₆ alkyl group, hydroxyalkyl group, hydrocarbyl or substituted hydrocarbyl group, alkoxylated group, benzyl group, or mixtures thereof; each R² is independently a C₁₄-C₂₂ alkyl group, hydroxyalkyl group, hydrocarbyl or substituted hydrocarbyl group, alkoxylated group, benzyl group, or mixtures thereof; and X⁻ is any quaternary ammonium-compatible anion.

In another example, the quaternary ammonium compounds may be mono or diester variations having the formula:
(R¹)₄₋ₘ—N+—[(CH₂)ₙ—Y—R³]ₘ X⁻
wherein:
Y is —O—(O)C—, or —C(O)—O—, or —NH—C(O)—, or —(O)—NH—; m is 1 to 3; n is 0 to 4; each R¹ is independently a C₁-C₆ alkyl group, hydroxyalkyl group, hydrocarbyl or substituted hydrocarbyl group, alkoxylated group, benzyl group, or mixtures thereof; each R³ is independently a C₁₃-C₂₁ alkyl group, hydroxyalkyl group, hydrocarbyl or substituted hydrocarbyl group, alkoxylated group, benzyl group, or mixtures thereof, and X⁻ is any quaternary ammonium-compatible anion.

In another example, the quaternary ammonium compound may be an imidazolinium compound, such as an imidazolinium salt.

As mentioned above, X⁻ can be any quaternary ammonium-compatible anion, for example, acetate, chloride, bromide, methyl sulfate, formate, sulfate, nitrate and the like can also be used in the present invention. In one example, X⁻ is chloride or methyl sulfate.

The surface treating composition may comprise additional ingredients such as a vehicle as described herein below which may not be present on the fibrous structure and/or sanitary tissue product comprising such fibrous structure. In one example, the surface treating composition may comprise a surface treating agent and a vehicle such as water to facilitate the application of the surface treating agent onto the surface of the fibrous structure.

Nonlimiting examples of quaternary ammonium compounds suitable for use in the present invention include the well-known dialkyldimethylammonium salts such as ditallowdimethylammonium chloride, ditallowdimethylammonium methylsulfate, di(hydrogenated tallow)dimethylammonium chloride. In one example, the surface treating composition comprises di(hydrogenated tallow)dimethylammonium chloride, commercially available from Witco Chemical Company Inc. of Dublin, Ohio as Varisoft 137®.

Nonlimiting examples of ester-functional quaternary ammonium compounds having the structures named above and suitable for use in the present invention include the well-known diester dialkyl dimethyl ammonium salts such as diester ditallow dimethyl ammonium chloride, monoester ditallow dimethyl ammonium chloride, diester ditallow dimethyl ammonium methyl sulfate, diester di(hydrogenated)tallow dimethyl ammonium methyl sulfate, diester di(hydrogenated)tallow dimethyl ammonium chloride, and mixtures thereof. In one example, the surface treating composition comprises diester ditallow dimethyl ammonium chloride and/or diester di(hydrogenated)tallow dimethyl ammonium chloride, both commercially available from Witco Chemical Company Inc. of Dublin, Ohio under the tradename “ADOGEN SDMC”.

Lotion Composition

The lotion composition may comprise oils and/or emollients and/or waxes and/or immobilizing agents. In one example, the lotion composition comprises from about 10% to about 90% and/or from about 30% to about 90% and/or from about 40% to about 90% and/or from about 40% to about 85% of an oil and/or emollient. In another example, the lotion composition comprises from about 10% to about 50% and/or from about 15% to about 45% and/or from about 20% to about 40% of an immobilizing agent. In another example, the lotion composition comprises from about 0% to about 60% and/or from about 5% to about 50% and/or from about 5% to about 40% of petrolatum.

The lotion compositions may be heterogeneous. They may contain solids, gel structures, polymeric material, a multiplicity of phases (such as oily and water phase) and/or emulsified components. It may be difficult to determine precisely the melting temperature of the lotion composition, i.e. difficult to determine the temperature of transition between the liquid form, the quasi-liquid from, the quasi-solid form and the solid form. The terms melting temperature, melting point, transition point and transition temperature are used interchangeably in this document and have the same meaning.

The lotion compositions may be semi-solid, of high viscosity so they do not substantially flow without activation during the life of the product or gel structures.

The lotion compositions may be shear thinning and/or they may strongly change their viscosity around skin temperature to allow for transfer and easy spreading on a user's skin.

The lotion compositions may be in the form of emulsions and/or dispersions.

In one example of a lotion composition, the lotion composition has a water content of less than about 20% and/or less than 10% and/or less than about 5% or less than about 0.5%.

In another example, the lotion composition may have a solids content of at least about 15% and/or at least about 25% and/or at least about 30% and/or at least about 40% to about 100% and/or to about 95% and/or to about 90% and/or to about 80%.

A nonlimiting example of a suitable lotion composition of the present invention comprises a chemical softening agent, such as an emollient, that softens, soothes, supples, coats, lubricates, or moisturizes the skin. The lotion composition may sooth, moisturize, and/or lubricate a user's skin.

The lotion composition may comprise an oil and/or an emollient. Nonlimiting examples of suitable oils and/or emollients include glycols (such as propylene glycol and/or glycerine), polyglycols (such as triethylene glycol), petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil (such as dimethicone) and mixtures thereof.

Nonlimiting examples of emollients useful in the present invention can be petroleum-based, fatty acid ester type, alkyl ethoxylate type, or mixtures of these materials. Suitable petroleum-based emollients include those hydrocarbons, or mixtures of hydrocarbons, having chain lengths of from 16 to 32 carbon atoms. Petroleum based hydrocarbons having these chain lengths include petrolatum (also known as “mineral wax,” “petroleum jelly” and “mineral jelly”). Petrolatum usually refers to more viscous mixtures of hydrocarbons having from 16 to 32 carbon atoms. A suitable Petrolatum is available from Witco, Corp., Greenwich, Conn. as White Protopet® 1 S.

Suitable fatty acid ester emollients include those derived from long chain C₁₂-C₂₈ fatty acids, such as C₁₆-C₂₂ saturated fatty acids, and short chain C₁-C₈ monohydric alcohols, such as C₁-C₃ monohydric alcohols. Nonlimiting examples of suitable fatty acid ester emollients include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈, such as C₁₂-C₁₆) and shorter chain fatty acids e.g., lactic acid, such as lauryl lactate and cetyl lactate.

Suitable fatty acid ester type emollients include those derived from C₁₂-C₂₈ fatty acids, such as C₁₆-C₂₂ saturated fatty acids, and short chain (C1-C₈ and/or C1-C₃) monohydric alcohols. Representative examples of such esters include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈ and/or C₁₂-C₁₆) and shorter chain fatty acids e.g., lactic acid, such as lauryl lactate and cetyl lactate.

Suitable alkyl ethoxylate type emollients include C₁₂-C₁₈ fatty alcohol ethoxylates having an average of from 3 to 30 oxyethylene units, such as from about 4 to about 23. Nonlimiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units), steareth-2 (a stearyl ethoxylate having an average of 2 oxyethylene units) and steareth-10 (a stearyl ethoxylate having an average of 10 oxyethylene units). These alkyl ethoxylate emollients are typically used in combination with the petroleum-based emollients, such as petrolatum, at a weight ratio of alkyl ethoxylate emollient to petroleum-based emollient of from about 1:1 to about 1:3, preferably from about 1:1.5 to about 1:2.5.

The lotion compositions of the present invention may include an “immobilizing agent”, so-called because they are believed to act to prevent migration of the emollient so that it can remain primarily on the surface of the fibrous structure to which it is applied so that it may deliver maximum softening benefit as well as be available for transferability to the user's skin. Suitable immobilizing agents for the present invention can comprise polyhydroxy fatty acid esters, polyhydroxy fatty acid amides, and mixtures thereof. To be useful as immobilizing agents, the polyhydroxy moiety of the ester or amide should have at least two free hydroxy groups. It is believed that these free hydroxy groups are the ones that co-crosslink through hydrogen bonds with the cellulosic fibers of the tissue paper web to which the lotion composition is applied and homo-crosslink, also through hydrogen bonds, the hydroxy groups of the ester or amide, thus entrapping and immobilizing the other components in the lotion matrix. Nonlimiting examples of suitable esters and amides will have three or more free hydroxy groups on the polyhydroxy moiety and are typically nonionic in character. Because of the skin sensitivity of those using paper products to which the lotion composition is applied, these esters and amides should also be relatively mild and non-irritating to the skin.

Suitable polyhydroxy fatty acid esters for use in the present invention will have the formula: 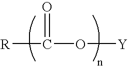
wherein R is a C₅-C₃, hydrocarbyl group, such as a straight chain C₇-C₁₉ alkyl or alkenyl and/or a straight chain C₉-C₁₇ alkyl or alkenyl and/or a straight chain C₁₁-C₁₇ alkyl or alkenyl, or mixture thereof; Y is a polyhydroxyhydrocarbyl moiety having a hydrocarbyl chain with at least 2 free hydroxyls directly connected to the chain; and n is at least 1. Suitable Y groups can be derived from polyols such as glycerol, pentaerythritol; sugars such as raffinose, maltodextrose, galactose, sucrose, glucose, xylose, fructose, maltose, lactose, mannose and erythrose; sugar alcohols such as erythritol, xylitol, malitol, mannitol and sorbitol; and anhydrides of sugar alcohols such as sorbitan.

One class of suitable polyhydroxy fatty acid esters for use in the present invention comprises certain sorbitan esters, such as sorbitan esters of C₁₆-C₂₂ saturated fatty acids.

Immobilizing agents include agents that are may prevent migration of the emollient into the fibrous structure such that the emollient remain primarily on the surface of the fibrous structure and/or sanitary tissue product and/or on the surface treating composition on a surface of the fibrous structure and/or sanitary tissue product and facilitate transfer of the lotion composition to a user's skin. Immobilizing agents may function as viscosity increasing agents and/or gelling agents.

Nonlimiting examples of suitable immobilizing agents include waxes (such as ceresin wax, ozokerite, microcrystalline wax, petroleum waxes, fisher tropsh waxes, silicone waxes, paraffin waxes), fatty alcohols (such as cetyl, cetaryl, cetearyl and/or stearyl alcohol), fatty acids and their salts (such as metal salts of stearic acid), mono and polyhydroxy fatty acid esters, mono and polyhydroxy fatty acid amides, silica and silica derivatives, gelling agents, thickeners and mixtures thereof.

In one example, the lotion composition comprises at least one immobilizing agent and at least one emollient.

Skin Benefit Agent

One or more skin benefit agents may be included in the lotion composition of the present invention. If a skin benefit agent is included in the lotion composition, it may be present in the lotion composition at a level of from about 0.5% to about 80% and/or 0.5% to about 70% and/or from about 5% to about 60% by weight of the lotion.

Nonlimiting examples of skin benefit agents include zinc oxide, vitamins, such as Vitamin B3 and/or Vitamin E, sucrose esters of fatty acids, such as Sefose 1618S (commercially available from Procter & Gamble Chemicals), antiviral agents, anti-inflammatory compounds, lipid, inorganic anions, inorganic cations, protease inhibitors, sequestration agents, chamomile extracts, aloe vera, calendula officinalis, alpha bisalbolol, Vitamin E acetate and mixtures thereof.

Nonlimiting examples of suitable skin benefit agents include fats, fatty acids, fatty acid esters, fatty alcohols, triglycerides, phospholipids, mineral oils, essential oils, sterols, sterol esters, emollients, waxes, humectants and combinations thereof.

In one example, the skin benefit agent may be any substance that has a higher affinity for oil over water and/or provides a skin health benefit by directly interacting with the skin. Suitable examples of such benefits include, but are not limited to, enhancing skin barrier function, enhancing moisturization and nourishing the skin.

The skin benefit agent may be alone, included in a lotion composition and/or included in a surface treating composition. A commercially available lotion composition comprising a skin benefit agent is Vaseline® Intensive Care Lotion (Chesebrough-Pond's, Inc.).

The lotion composition may be a transferable lotion composition. A transferable lotion composition comprises at least one component that is capable of being transferred to an opposing surface such as a user's skin upon use. In one example, at least 0.1% of the transferable lotion present on the user contacting surface transfers to the user's skin during use.

Other Ingredients

Other optional ingredients that may be included in the lotion composition include vehicles, perfumes, especially long lasting and/or enduring perfumes, antibacterial actives, antiviral actives, disinfectants, pharmaceutical actives, film formers, deodorants, opacifiers, astringents, solvents, cooling sensate agents, such as camphor, thymol and menthol.

Vehicle

As used herein a “vehicle” is a material that can be used to dilute and/or emulsify agents forming the surface treating composition and/or lotion composition to form a dispersion/emulsion. A vehicle may be present in the surface treating composition and/or lotion composition, especially during application of the surface treating composition and/or to the fibrous structure. A vehicle may dissolve a component (true solution or micellar solution) or a component may be dispersed throughout the vehicle (dispersion or emulsion). The vehicle of a suspension or emulsion is typically the continuous phase thereof. That is, other components of the dispersion or emulsion are dispersed on a molecular level or as discrete particles throughout the vehicle.

Suitable materials for use as the vehicle of the present invention include hydroxyl functional liquids, including but not limited to water. In one example, the lotion composition comprises less than about 20% and/or less than about 10% and/or less than about 5% and/or less than about 0.5% w/w of a vehicle, such as water. In one example, the surface treating composition comprises greater than about 50% and/or greater than about 70% and/or greater than about 85% and/or greater than about 95% and/or greater than about 98% w/w of a vehicle, such as water.

Process Aids

Process aids may also be used in the lotion compositions of the present invention. Nonlimiting examples of suitable process aids include brighteners, such as TINOPAL CBS-X®, obtainable from CIBA-GEIGY of Greensboro, N.C.

Nonlimiting Examples of Lotion Compositions

### EXAMPLE 1 OF LOTION COMPOSITION

| | |
|---|---|
| Stearyl Alcohol CO1897 * | 40% w/w |
| Petrolatum Snowwhite V28EP ** | 30% w/w |
| Mineral oil Carnation ** | 30% w/w |

* Available from Procter&Gamble; Chemicals, Cincinnati, USA.
** Available from Witco.

The lotion composition has a melting point of about 51° C. and a melt viscosity at 56° C. of about 17 m*Pas measured at a shear rate of 0.1 1/s. The mineral oil used in this formulation has a viscosity of about 21 mpa*s at 20° C.

### EXAMPLE 2 OF LOTION COMPOSITION

| | |
|---|---|
| Mineral oil * | 55% w/w |
| Paraffin ** | 12% w/w |
| Cetaryl alcohol | 21% w/w |
| Steareth-2 *** | 11% w/w |
| Skin benefit agent | 1% w/w |

* Drakeol 7PG available from Penreco.
** Chevron 128 available from Chevron.
*** Available from Abitec Corporation.

All documents cited in the Detailed Description of the Invention are, in relevant part, incorporated by reference herein; the citation of any document is not to be construed as an admission that it is prior art with respect to the present invention. To the extent that any meaning or definition of the term in this written document conflicts with any meaning or definition of the term in a document incorporated by reference, the meaning or definition assigned to the term in this written document shall govern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fibrous structure comprising a surface comprising two or more different compositions wherein at least two of the two or more different compositions are phase registered.

2. The fibrous structure according to claim 1 wherein at least one of the two or more different compositions comprises a surface treating composition.

3. The fibrous structure according to claim 1 wherein at least one of the two or more different compositions comprises a lotion composition.

4. The fibrous structure according to claim 1 wherein at least one of the two or more different compositions comprises a surface treating composition and at least one of the two or more different compositions comprises a lotion composition, wherein the surface treating composition is positioned between a lotion composition and the surface of the fibrous structure.

5. The fibrous structure according to claim 1 wherein the two or more different compositions are present on the surface of the fibrous structure in two or more different layers.

6. The fibrous structure according to claim 1 wherein the two or more different compositions comprise at least two incompatible compositions.

7. The fibrous structure according to claim 1 wherein the surface comprises a first region comprising the two or more different compositions and a second region that is void of at least one of the two or more different compositions.

8. A single- or multi-ply sanitary tissue product comprising a fibrous structure according to claim 1.

9. A fibrous structure comprising a surface comprising:
a. a first region comprising a surface treating composition and a lotion composition; and
b. a second region being void of at least one of the surface treating composition and the lotion composition;
wherein the second region is surrounded by the first region.

10. The fibrous structure according to claim 9 wherein the lotion composition is phase registered with the surface treating composition.

11. A single- or multi-ply sanitary tissue product comprising a fibrous structure according to claim 9.

12. A fibrous structure comprising a surface comprising a first composition and a second composition that is incompatible with the first composition, wherein the first and second compositions are phase registered.

13. The fibrous structure according to claim 12 wherein the surface of the fibrous structure further comprises discrete regions void of at least one of the first and second compositions.

14. The fibrous structure according to claim 13 wherein the phase registered first and second compositions are present on the surface in the form of a continuous network.

15. The fibrous structure according to claim 12 wherein the phase registered first and second compositions are present on the surface in the form of discrete regions.

16. The fibrous structure according to claim 15 wherein the surface further comprises a continuous network void of at least one of the first and second compositions.

17. The fibrous structure according to claim 12 wherein the fibrous structure is a through-air-dried fibrous structure.

18. The fibrous structure according to claim 12 wherein the fibrous structure is a conventionally dried fibrous structure.

19. A single- or multi-ply sanitary tissue product comprising a fibrous structure according to claim 12.

20. A process for treating a surface of a fibrous structure, the process comprising the steps of:
a. applying a first composition to the surface of the fibrous structure;
b. applying a second composition to the first composition; and
c. applying a third composition to the second composition.

21. The process according to claim 20 wherein the steps of applying a first composition and applying a second composition occur sequentially.

22. The process according to claim 20 wherein the steps of applying a first composition and applying a second composition is performed by a single device.

23. The process according to claim 20 wherein at least two of the first, second and third compositions are incompatible.

24. A process for treating a surface of a fibrous structure, the process comprising the steps of:
a. applying a first composition to the surface of the fibrous structure; and
b. phase registrably applying a second composition to the first composition.

25. The process according to claim 24 wherein the first composition and second composition are incompatible.

26. The process according to claim 24 wherein the process further comprises a step of applying a third composition to the second composition.
